(19) United States
(12) Patent Application Publication
(10) Pub. No.: US 2021/0104070 A1
(43) Pub. Date: Apr. 8, 2021

(54) IMAGE PROCESSING APPARATUS, IMAGE PROCESSING METHOD, AND COMPUTER-READABLE RECORDING MEDIUM

(71) Applicant: OLYMPUS CORPORATION, Tokyo (JP)

(72) Inventor: Masanori MITSUI, Tokyo (JP)

(73) Assignee: OLYMPUS CORPORATION, Tokyo (JP)

(21) Appl. No.: 17/117,338

(22) Filed: Dec. 10, 2020

Related U.S. Application Data
(63) Continuation of application No. PCT/JP2018/022625, filed on Jun. 13, 2018.

(51) Int. Cl. G06T 7/90; G06K 9/62; G06K 9/46; G06K 9/54; G06T 7/00; G06T 11/00; G06N 5/04; G06N 20/00

(52) U.S. Cl. CPC G06T 7/90; G06K 9/6267; G06K 9/4652; G06K 9/54; G06K 9/6217; G06T 7/0012; G06T 2207/10056; G06N 5/04; G06N 20/00; G06T 2207/20212; G06T 2207/20081; G06K 2209/05; G06T 11/001

(57) ABSTRACT

An image processing apparatus includes a processor including hardware. The processor is configured to: calculate a hue of each pixel of a stained image that is input from outside; execute classification on each pixel of the stained image based on the hue; modulate a color tone of the pixel of the stained image in each class having undergone the classification; combine a plurality of input images to generate a combined image; calculate a color distribution of each pixel of the combined image; execute classification on each pixel of the combined image by using the color distribution; and calculate an average hue of each class having undergone the classification on each pixel of the combined image based on the color distribution and a classification result of classification of the combined image so as to calculate a standard hue.

### CROSS-REFERENCE TO RELATED APPLICATION

This application is a continuation of International Application No. PCT/JP2018/022625, filed on Jun. 13, 2018, the entire contents of which are incorporated herein by reference.

### BACKGROUND

### 1. Technical Field

The present disclosure relates to an image processing apparatus and, more particularly, to an image processing apparatus that executes image processing on a microscopic image of a pathologic specimen, and relates to an image processing method and a computer-readable recording medium.

### 2. Related Art

In the relater art, for the diagnosis on a living tissue specimen including a pathologic specimen, a block specimen obtained by organ harvesting or a specimen obtained by needle biopsy is sliced in the thickness of approximately several microns, and the observation image obtained by enlarging the sliced specimen with a microscope is observed. Transmission observation using an optical microscope is one of the traditional and the most popular observation techniques with low costs of a device and easy handling. In recent years, diagnosis has been conducted by using the image obtained by taking the observation image using an imaging device attached to an optical microscope.

A sliced living tissue specimen (hereinafter referred to as “sliced specimen”) hardly absorbs or scatters light and is almost colorless and transparent. Therefore, typically, a sliced specimen is stained prior to microscopy.

Various dyeing techniques are disclosed, and the total number thereof reaches 100 or more types. Among the staining techniques, hematoxylin-eosin stain (hereinafter referred to as “HE stain”) using two dyes, blue-violet hematoxylin (hereinafter simply referred to as “H”) and red eosin (hereinafter simply referred to as “E”), is normally used for pathologic specimens in particular.

In the clinical practice, when it is difficult to visually recognize a living tissue that is the target to be observed with HE stain or when the morphological diagnosis of a living tissue is interpolated, a technique may be sometimes used to apply a special stain different from the HE stain to a specimen and change the color of the target tissue to be observed so as to visually highlight the target tissue. Further, in histopathological diagnosis, immunostaining (immunohistochemistry: IHC) using various marker proteins for visualizing, for example, antigen-antibody reaction of a cancer tissue is sometimes used.

Observation of a stained specimen is executed by displaying, on a display device, the image generated by capturing the stained specimen with an imaging device as well as by visual recognition. In recent years, there has been the proposed attempt to execute image processing on a stained specimen image generated by capturing with an imaging device and conduct analysis so as to support the observation and diagnosis by a doctor, etc. For this analysis, there is a technique using learning such as deep learning. In this case, the calculated parameter is obtained by learning the pair including an analysis value corresponding to the RGB value of an input image.

However, in the observation of a stained specimen, even the tissues in the same condition may have different color shades because of a difference in the color due to the capturing state with regard to the stained specimen, a difference in the color due to a dyeing process, for example, a difference in the spectrum of a dye, or a difference in the staining time. In deep learning, or the like, when the color shade of an input image is different from the color shade of a learning image, the inference accuracy is degraded. For this reason, in deep learning, or the like, it is possible to deal with a larger number of color shades of a learning image; however, it is not practical as an enormous number of images are required under various conditions. Therefore, there is a known technique for performing color equalization to correct different color shades to the identical color shade (see Japanese Patent No. 5137481).

### SUMMARY

In some embodiments, an image processing apparatus includes a processor comprising hardware. The processor is configured to: calculate a hue of each pixel of a stained image that is input from outside; execute classification on each pixel of the stained image based on the hue; modulate a color tone of the pixel of the stained image in each class having undergone the classification; combine a plurality of input images to generate a combined image; calculate a color distribution of each pixel of the combined image; execute classification on each pixel of the combined image by using the color distribution; and calculate an average hue of each class having undergone the classification on each pixel of the combined image based on the color distribution and a classification result of classification of the combined image so as to calculate a standard hue.

In some embodiments, an image processing apparatus includes a processor comprising hardware. The processor is configured to: calculate a hue of each pixel of a stained image that is input from outside; execute classification on each pixel of the stained image based on the hue; modulate a color tone of the pixel of the stained image in each class having undergone the classification; rotate a hue of an input image associated with a correct value at different rotation angles to generate a plurality of images having different hues; generate, based on the plurality of images and on a learning result stored in a storage, a plurality of output images; combine hue ranges of images whose error between the plurality of output images and the correct value falls within an allowable range to calculate a color distribution; and calculate an average hue of each class having undergone classification on each pixel of the input image by using the color distribution so as to calculate a standard hue.

In some embodiments, provide is an image processing method implemented by an image processing apparatus. The image processing method includes: calculating a hue of each pixel of a stained image that is input from outside; executing classification on each pixel of the stained image based on the hue; modulating a color tone of the pixel of the stained image in each class having undergone the classification; combining a plurality of input images to generate a combined image; calculating a color distribution of each pixel of the combined image; executing classification on each pixel of the combined image by using the color distribution; and calculating an average hue of each class having undergone the classification on each pixel of the combined image based on the color distribution and a classification result of classification of the combined image so as to calculate a standard hue.

In some embodiments, provided is a non-transitory computer-readable recording medium with an executable program stored thereon. The program causes an image processing apparatus to execute: calculating a hue of each pixel of a stained image that is input from outside; executing classification on each pixel of the stained image based on the hue; modulating a color tone of the pixel of the stained image in each class having undergone the classification; combining a plurality of input images to generate a combined image; calculating a color distribution of each pixel of the combined image; executing classification on each pixel of the combined image by using the color distribution; and calculating an average hue of each class having undergone the classification on each pixel of the combined image based on the color distribution and a classification result of classification of the combined image so as to calculate a standard hue.

In some embodiments, provided is an image processing method implemented by an image processing apparatus. The image processing method includes: calculating a hue of each pixel of a stained image that is input from outside; executing classification on each pixel of the stained image based on the hue; modulating a color tone of the pixel of the stained image in each class having undergone the classification; rotating a hue of an input image associated with a correct value at different rotation angles to generate a plurality of images having different hues; generating, based on the plurality of images and on a learning result stored in a storage, a plurality of output images; combining hue ranges of images whose error between the plurality of output images and the correct value falls within an allowable range to calculate a color distribution; and calculating an average hue of each class having undergone classification on each pixel of the input image by using the color distribution so as to calculate a standard hue.

In some embodiments, provided is a non-transitory computer-readable recording medium with an executable program stored thereon. The program causes an image processing apparatus to execute: calculating a hue of each pixel of a stained image that is input from outside; executing classification on each pixel of the stained image based on the hue; modulating a color tone of the pixel of the stained image in each class having undergone the classification; rotating a hue of an input image associated with a correct value at different rotation angles to generate a plurality of images having different hues; generating, based on the plurality of images and on a learning result stored in a storage, a plurality of output images; combining hue ranges of images whose error between the plurality of output images and the correct value falls within an allowable range to calculate a color distribution; and calculating an average hue of each class having undergone classification on each pixel of the input image by using the color distribution so as to calculate a standard hue.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a functional configuration of an image processing apparatus according to a first embodiment of the present disclosure;

FIG. 2 is a flowchart illustrating the overview of a process performed by the image processing apparatus according to the first embodiment of the present disclosure;

FIG. 3 is a diagram schematically illustrating a reference hue parameter;

FIG. 4 is a diagram schematically illustrating a hue distribution of an input image;

FIG. 5 is a diagram schematically illustrating the relationship between saturation and a hue angle;

FIG. 6 is a diagram schematically illustrating a hue distribution of the input image after hue rotation;

FIG. 7 is a block diagram illustrating a functional configuration of an image processing apparatus according to a second embodiment of the present disclosure;

FIG. 8 is a flowchart illustrating the overview of a process performed by the image processing apparatus according to the second embodiment of the present disclosure;

FIG. 9 is a block diagram illustrating a functional configuration of the image processing apparatus according to a third embodiment of the present disclosure;

FIG. 10 is a flowchart illustrating the overview of a process performed by the image processing apparatus according to the third embodiment of the present disclosure;

FIG. 11 is a diagram schematically illustrating a reference hue parameter;

FIG. 12 is a diagram schematically illustrating a hue distribution of an input image;

FIG. 13 is a diagram schematically illustrating a hue distribution after setting of a fixed value for the hue of the input image;

FIG. 14 is a block diagram illustrating a functional configuration of an image processing apparatus according to a fourth embodiment of the present disclosure;

FIG. 15 is a flowchart illustrating the overview of a process performed by the image processing apparatus according to the fourth embodiment of the present disclosure;

FIG. 16 is a diagram schematically illustrating an example of an image displayed by a display unit;

FIG. 17 is a diagram schematically illustrating an example of another image displayed by the display unit;

FIG. 18 is a block diagram illustrating a functional configuration of an image processing apparatus according to a fifth embodiment of the present disclosure;

FIG. 19 is a flowchart illustrating the overview of a process performed by the image processing apparatus according to the fifth embodiment of the present disclosure;

FIG. 20 is a diagram schematically illustrating an example of images input to an input unit;

FIG. 21 is a diagram schematically illustrating an example of a standard distribution by a standard-hue calculator;

FIG. 22 is a diagram schematically illustrating an average hue axis;

FIG. 23 is a diagram schematically illustrating an example of an input image to be learned by a learning unit;

FIG. 24 is a diagram schematically illustrating an example of a correct image to be learned by the learning unit; and

FIG. 25 is a diagram schematically illustrating a learning process by the learning unit.

### DETAILED DESCRIPTION

An image processing apparatus, an image processing method, and a program according to embodiments of the present disclosure are described below with reference to the drawings. The present disclosure is not limited to the embodiments. In the descriptions of the drawings, the same parts are denoted by the same reference numeral.

### First Embodiment

Configuration of Image Processing Apparatus

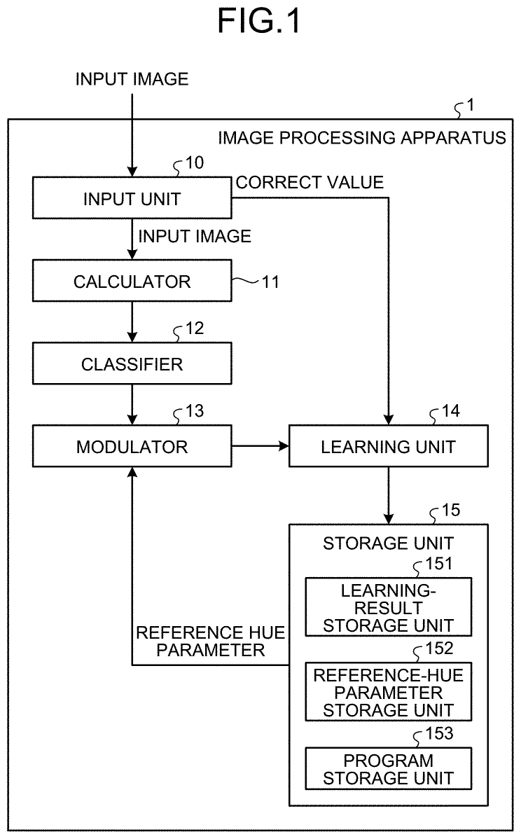

FIG. 1 is a block diagram illustrating a functional configuration of an image processing apparatus according to a first embodiment. An image processing apparatus 1 illustrated in FIG. 1 is, for example, an apparatus that executes image processing to modulate the hue of a stained image, which is acquired by capturing a stained specimen with a microscope or a video microscope, for simple color normalization so as to suppress color variations of a training image that is an input image (stained image) used for machine learning. A stained image and a training image are normally a color image having a pixel level (pixel value) corresponding to the wavelength components of R (red), G (green), and B (blue) at each pixel position.

Hereinafter, a stained image is an image obtained by capturing a specimen that is stained by using, for example, HE stain, Masson's trichrome stain, Papanicolaou stain, or immunostaining. HE stain is used for typical tissue morphological observation to stain a nucleus in blue violet (hematoxylin) and cytoplasm in pink (eosin). Masson's trichrome stain is to stain a collagen fiber in blue (aniline blue), a nucleus in black violet, and cytoplasm in red. Papanicolaou stain is used for cell examination to stain cytoplasm in orange, light green, or the like, depending on the degree of differentiation. Immunostaining is used for immune antibody reaction to stain specific tissues. Specifically, immunostaining causes the antibody to combine with the DAB dye to stain a nucleus with hematoxylin. In the description according to the embodiments below, the input image is the image obtained by capturing a specimen that is stained by immunostaining; however, changes may be made as appropriate depending on a staining technique.

The image processing apparatus 1 illustrated in FIG. 1 includes an input unit 10, a calculator 11, a classifier 12, a modulator 13, a learning unit 14, and a storage unit 15.

The input unit 10 receives the learning data in which an input image, input from outside the image processing apparatus 1, is associated with a correct value. The input unit 10 outputs an input image (training image) included in the learning data to the calculator 11 and outputs a correct value to the learning unit 14. The input unit 10 is configured by using, for example, an interface module capable of communicating bi-directionally with the outside.

The calculator 11 calculates the hue of the input image that is input from the input unit 10 in each pixel of the input image, and outputs the calculated hue of the input image in each pixel of the input image and the input image that is input from the input unit 10, to the classifier 12. The calculator 11 may divide the input image into predetermined regions and calculate the hue of the input image in each divided region.

The classifier 12 executes classification on each pixel or predetermined region of the input image, input from the calculator 11, based on the hue of the input image in each pixel input from the calculator 11 and outputs the classification result and the input image that is input from the calculator 11, to the modulator 13.

The modulator 13 modulates the color tone of the pixel of the input image in each class, which has undergone the classification and input from the classifier 12, and outputs the modulation result to the learning unit 14. Specifically, based on a reference hue parameter in the storage unit 15 described below, the modulator 13 modulates the hue of each image in each class, which has undergone the classification and input from the classifier 12, and outputs the input image with the modulated hue to the learning unit 14.

The learning unit 14 executes machine learning such as regression analysis or a neural network based on the input image with the modulated hue, input from the modulator 13, and on the correct value associated with the input image and stores the learning result in a learning-result storage unit 151 of the storage unit 15. The targets for learning by the learning unit 14 are various, including for example the one for estimating the amount of dye, the one for executing tissue classification, and the one for determining the grade of a disease state (lesion). The correct value is the image having the quantitative values corresponding to the number of dyes for each pixel in the case of the amount of dye, is the class number assigned to each pixel in the case of tissue distribution, and is the value indicating a single grade and assigned to a single image in the case of the grade of a disease state.

The storage unit 15 is configured by using a volatile memory, a non-volatile memory, a memory card, or the like. The storage unit 15 includes the learning-result storage unit 151, a reference-hue parameter storage unit 152, and a program storage unit 153. The learning-result storage unit 151 stores a learning result obtained by learning of the learning unit 14. The reference-hue parameter storage unit 152 stores the reference hue parameter that is referred to when the modulator 13 modulates the hue of a training image. The program storage unit 153 stores various programs executed by the image processing apparatus 1 and various types of data used during the execution of a program.

The image processing apparatus 1 having the above configuration is configured by using, for example, a central processing unit (CPU), a graphics processing unit (GPU), a field programmable gate array (FPGA), or a digital signal processing (DSP) to read various programs from the program storage unit 153 of the storage unit 15 and send an instruction or data to each unit included in the image processing apparatus 1 so as to perform each function.

Process of Image Processing Apparatus

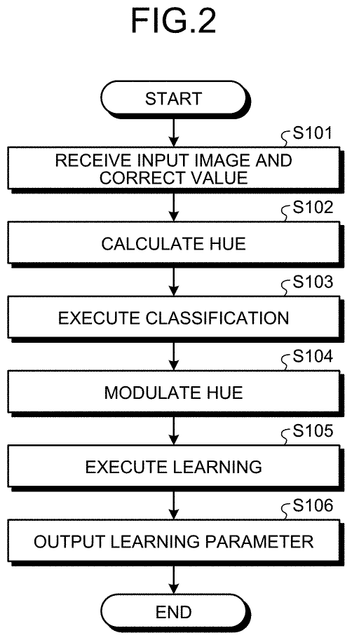

Next, a process performed by the image processing apparatus 1 is described. FIG. 2 is a flowchart illustrating the overview of a process performed by the image processing apparatus 1.

As illustrated in FIG. 2, the input unit 10 first receives an input image and a correct value from outside (Step S101). In this case, the input unit 10 outputs the input image, input from outside, to the calculator 11 and outputs the correct value to the learning unit 14.

Then, the calculator 11 calculates the hue of each pixel of the input image, input from the input unit 10 (Step S102). Specifically, the calculator 11 calculates the hue of each pixel of the input image and outputs the calculation result to the classifier 12.

Then, the classifier 12 executes classification on each pixel of the input image based on the hue of each pixel of the input image calculated by the calculator 11 (Step S103). Specifically, the classifier 12 classifies each pixel of the input image into a DAB pixel, an H pixel, or other pixels based on the hue calculated by the calculator 11 and outputs a result of the classification to the modulator 13.

Subsequently, the modulator 13 modulates, based on the reference hue parameter, the hue of the pixel of the input image in each classification input from the classifier 12 (Step S104). Specifically, the modulator 13 executes hue modulation on a DAB pixel and an H pixel, which have undergone the classification and input from the classifier 12, based on the reference hue parameter stored in the reference-hue parameter storage unit 152 and does not execute hue modulation on other pixels. After Step S104, the image processing apparatus 1 proceeds to Step S105 described below.

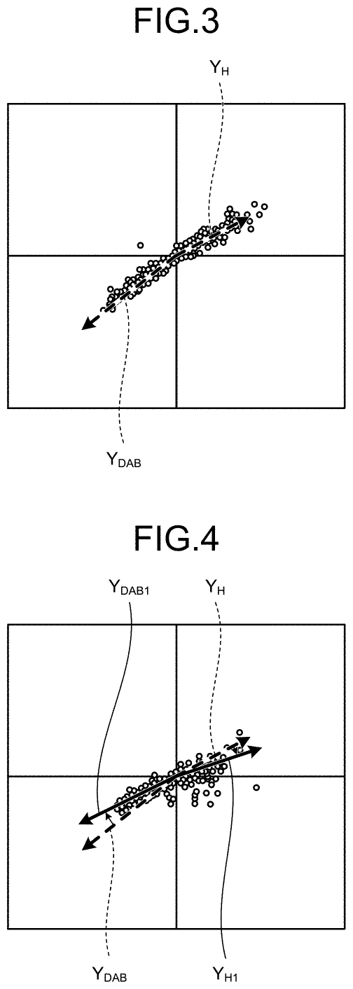

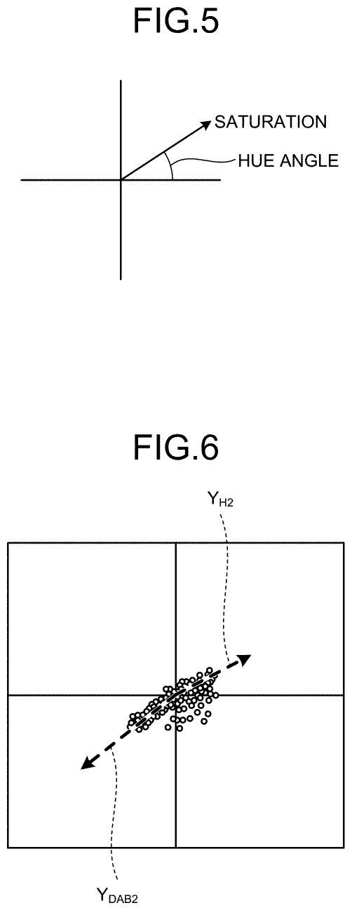

Here, the details of a hue modulation process executed by the modulator 13 is described. FIG. 3 is a diagram schematically illustrating the reference hue parameter. FIG. 4 is a diagram schematically illustrating the hue distribution of an input image. FIG. 5 is a diagram schematically illustrating the relationship between saturation and a hue angle. FIG. 6 is a diagram schematically illustrating the hue distribution of the input image after hue rotation. FIGS. 3, 4, and 6 illustrate an example of two dyes, DAB and H. Furthermore, FIGS. 3, 4, and 6 illustrate the hue distribution by using the a*b* plane. FIGS. 3, 4, and 6 illustrate each pixel using a single dot. In FIG. 3, an arrow Y_{H} represents an H-color hue axis of the standard hue parameter, and an arrow YA represents a DAB-color hue axis of the standard hue parameter. In FIG. 4, an arrow Y_{H1} represents the H-color hue axis of the standard hue parameter, and an arrow Y_{DAB1} represents the DAB-color hue axis of the standard hue parameter.

As illustrated in FIG. 3, the reference hue parameter includes two average hues, the DAB average hue and the H average hue. As illustrated in FIG. 4, the modulator 13 first calculates the DAB average hue and the H average hue based on the distribution of the hue calculated by the calculator 11. Specifically, as indicated by the arrow Y_{H1} and the arrow Y_{DAB1} the modulator 13 calculates the average value of the hues of the pixels within the hue range that is previously set for each dye. Then, as illustrated in FIGS. 5 and 6, the modulator 13 rotates the hue of each pixel of the input image so that the arrow Y_{H1} and the arrow Y_{DAB1} match the arrow Y_{H} and the arrow Y_{DAB}, respectively, of the reference hue parameter. In this manner, the modulator 13 rotates the hue of the input image such that the average hue of the pixels of the input image matches the reference hue of the reference parameter. The modulator 13 converts the RGB value of each pixel of the input image into the hue, the luminance, and the saturation of the HLS color space and, among them, modulates the hue signal of the hue. As the method for modulating the hue, the modulator 13 may execute rotation on a color-difference signal plane after division into a luminance signal and a color-difference signal, such as the a*b plane of the L*a*b in the Lab color space other than the HLS color space.

With reference back to FIG. 2, the description of Step S105 and subsequent steps is continued.

The learning unit 14 executes learning by using the pair of the training image with the modulated hue, input from the modulator 13, and the correct value input from the input unit 10 (Step S105) and outputs a learning parameter that is a learning result to the learning-result storage unit 151 (Step S106). After Step S106, the image processing apparatus 1 ends this process.

According to the first embodiment described above, the hue of the input image is modulated so as to match the color shade; therefore, even when there are color variations due to a difference in stains, there is no need to execute learning of an input image for each stain, and different learning images may be acquired in a simple process, which enables effective learning.

### Second Embodiment

Next, a second embodiment of the present disclosure is described. According to the second embodiment, after the hue of an input image is modulated, inference is executed by using a learning result. After the configuration of an image processing apparatus according to the second embodiment is described, a process performed by the image processing apparatus according to the second embodiment is described below. The same components as those of the image processing apparatus 1 according to the first embodiment described above are denoted by the same reference numeral, and the detailed description is omitted.

Configuration of Image Processing Apparatus

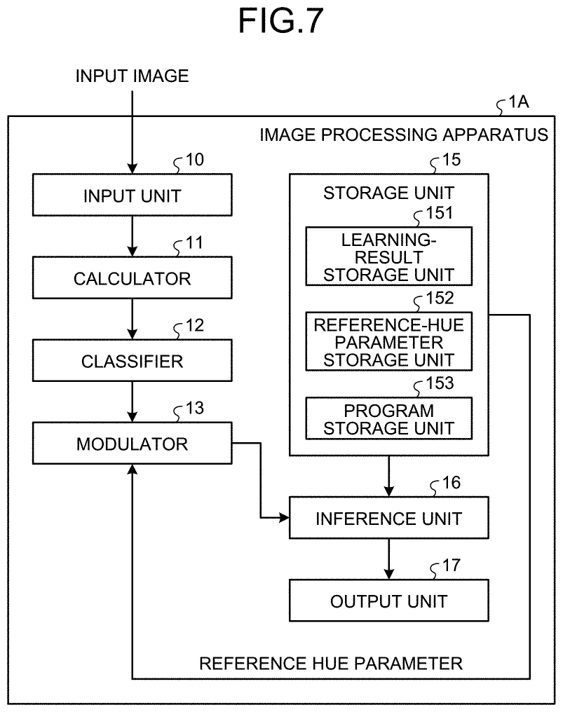

FIG. 7 is a block diagram illustrating a functional configuration of the image processing apparatus according to the second embodiment. An image processing apparatus 1A illustrated in FIG. 7 includes the input unit 10, the calculator 11, the classifier 12, the modulator 13, the storage unit 15, an inference unit 16, and an output unit 17.

The inference unit 16 executes inference based on a learning result stored in the learning-result storage unit 151 and a training image input from the modulator 13 and outputs the inference result to the output unit 17.

The output unit 17 outputs the inference result input from the inference unit 16. The output unit 17 is configured by using, for example, a display panel of a liquid crystal or an organic electro luminescence (EL) or a speaker. It is obvious that the output unit 17 may be configured by using an output interface module that outputs an inference result to an external display device, etc.

Process of Image Processing Apparatus

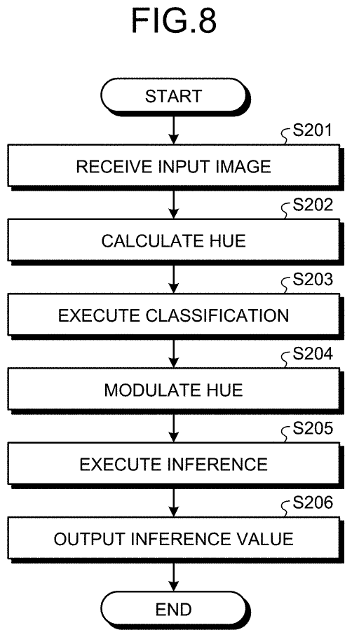

Next, a process performed by the image processing apparatus 1A is described. FIG. 8 is a flowchart illustrating the overview of the process performed by the image processing apparatus 1A. In FIG. 8, Steps S201 to S204 correspond to Steps S101 to S104, respectively, in FIG. 2 described above.

At Step S205, the inference unit 16 applies a learning parameter, which is a learning result stored in the learning-result storage unit 151, to the modulated training image input from the modulator 13 to execute inference. In this case, the inference unit 16 outputs the inference result (inference value) to the output unit 17.

Subsequently, the output unit 17 outputs the inference value input from the inference unit 16 (Step S206).

According to the second embodiment described above, as the hue of the input training image is modulated so as to match the color shade, it is possible to input the image having the same color shade as that used for learning, which enables high-accuracy inference.

### Third Embodiment

Next, a third embodiment of the present disclosure is described. According to the third embodiment, learning is executed by selectively using hue rotation and fixing for each class. After a configuration of an image processing apparatus according to the third embodiment is described, a process performed by the image processing apparatus according to the third embodiment is described below. The same components as those of the image processing apparatus 1A according to the second embodiment described above are denoted by the same reference numeral, and the detailed description is omitted.

Configuration of Image Processing Apparatus

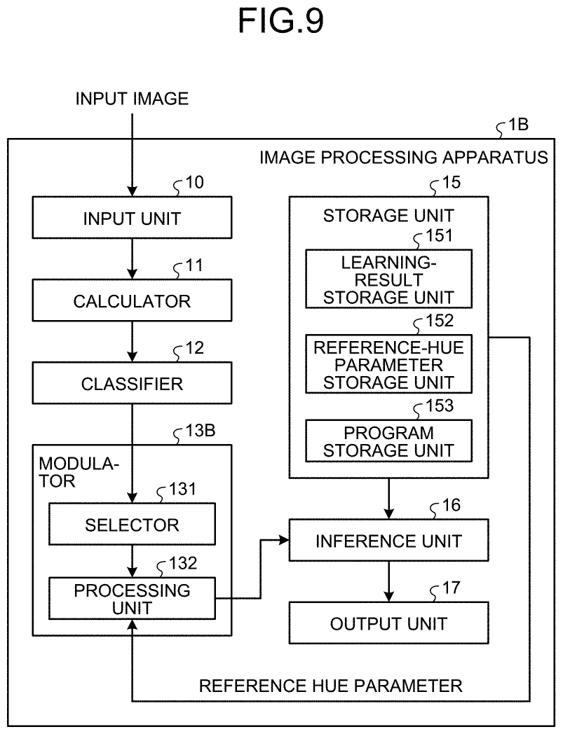

FIG. 9 is a block diagram illustrating a functional configuration of the image processing apparatus according to the third embodiment. An image processing apparatus 1B illustrated in FIG. 9 includes a modulator 13B instead of the modulator 13 according to the second embodiment described above. The modulator 13B includes a selector 131 and a processing unit 132.

The selector 131 selects and determines the method for modulating a hue for each class input from the classifier 12 and outputs the determination result, the input image, and the classification result to the processing unit 132.

With regard to the input image that is input from the selector 131, the processing unit 132 modulates the hue of each class by using the modulation method selected by the selector 131 for each class and outputs the input image with the modulated hue to the inference unit 16.

Process of Image Processing Apparatus

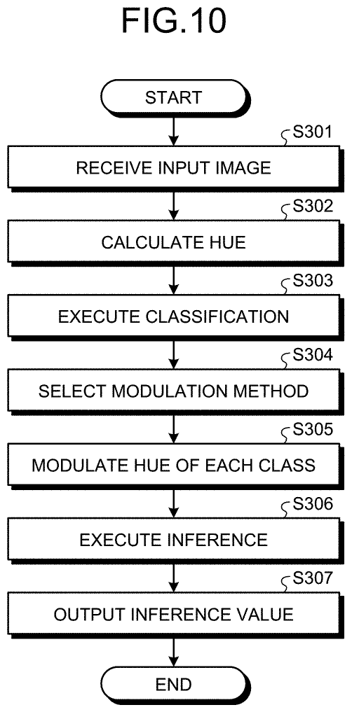

Next, a process performed by the image processing apparatus 1B is described. FIG. 10 is a flowchart illustrating the overview of the process performed by the image processing apparatus 1B. In FIG. 10, Steps S301 to S303, S306, and S307 correspond to Steps S201 to S203, S205, and S206, respectively, in FIG. 8 described above.

At Step S304, the selector 131 selects the method for modulating the hue for each class input from the classifier 12. Specifically, when two dyes, DAB and H are used, and a classification is made into each dye, a DAB class and an H class, the selector 131 selects the modulation method to rotate the hue so as to leave the original distribution as DAB needs a fixed value. The selector 131 selects the method for modulating the hue by using the fixed value for the hue as the shape identification is only necessary for H.

Subsequently, with regard to the input image that is input from the selector 131, the processing unit 132 modulates the hue of each class by using the modulation method selected by the selector 131 for each class and outputs the input image with the modulated hue to the inference unit 16 (Step S305). After Step S305, the image processing apparatus 1B proceeds to Step S306.

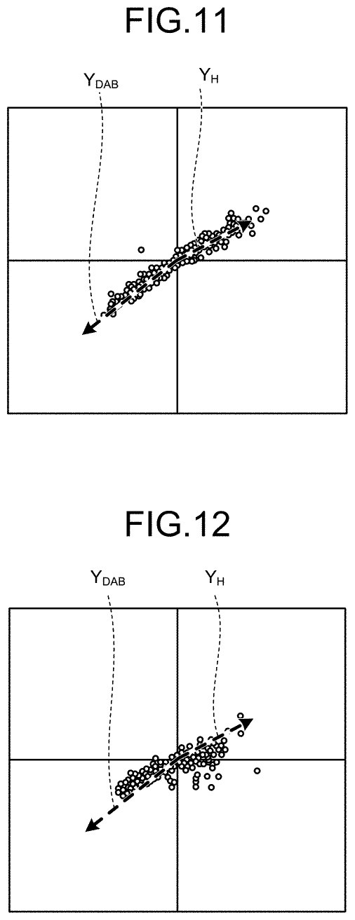

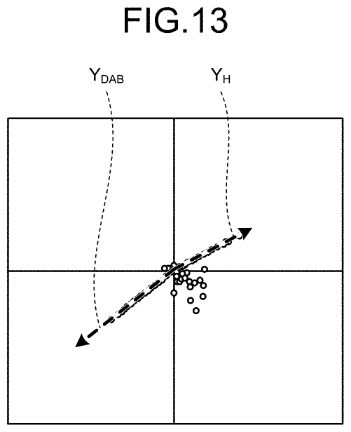

The details of a hue modulation process performed by the processing unit 132 are described. FIG. 11 is a diagram schematically illustrating a reference hue parameter. FIG. 12 is a diagram schematically illustrating the hue distribution of an input image. FIG. 13 is a diagram schematically illustrating the hue distribution after setting of the fixed value for the hue of the input image. FIGS. 11 to 13 illustrate an example of two dyes, DAB and H. FIGS. 11 to 13 illustrates the hue distribution by using the a*b* plane. FIGS. 11 to 13 illustrate each pixel using a single dot. In FIGS. 11 to 13, the arrow Y_{H} represents the H-color hue axis of the standard hue parameter, and the arrow Y_{DAB} represents the DAB-color hue axis of the standard hue parameter. In FIGS. 11 to 13, the H-color hue axis and the DAB-color hue axis of the standard hue parameter have fixed values.

As illustrated in FIGS. 11 to 13, the processing unit 132 rotates the hue so as to leave the original distribution as DAB needs the fixed value based on the modulation method selected by the selector 131. The processing unit 132 changes the hue such that the hue has the fixed value as the shape identification is only necessary for H. Specifically, as illustrated in FIGS. 11 to 13, the processing unit 132 uses the H-color hue axis and the DAB-color hue axis of the standard hue parameter as fixed values and modulates the hue value into the reference hue for each corresponding class of each pixel of the input image. As a result, as illustrated in FIG. 13, the hue distribution after the setting of the fixed value of the hue is a linear distribution having the same value in each class.

According to the third embodiment described above, as the hue distribution after the setting of the fixed value of the hue is a linear distribution having the same value in each class, it is possible to input the image having the same color shade as that used for learning, which enables high-accuracy inference.

### Fourth Embodiment

Next, a fourth embodiment of the present disclosure is described. According to the fourth embodiment, hue modulation is executed so that different color shades of the images are changed to the same color shade for observation. The same components as those of the image processing apparatus 1A according to the second embodiment described above are denoted by the same reference numeral, and detailed description is omitted.

Configuration of Image Processing Apparatus

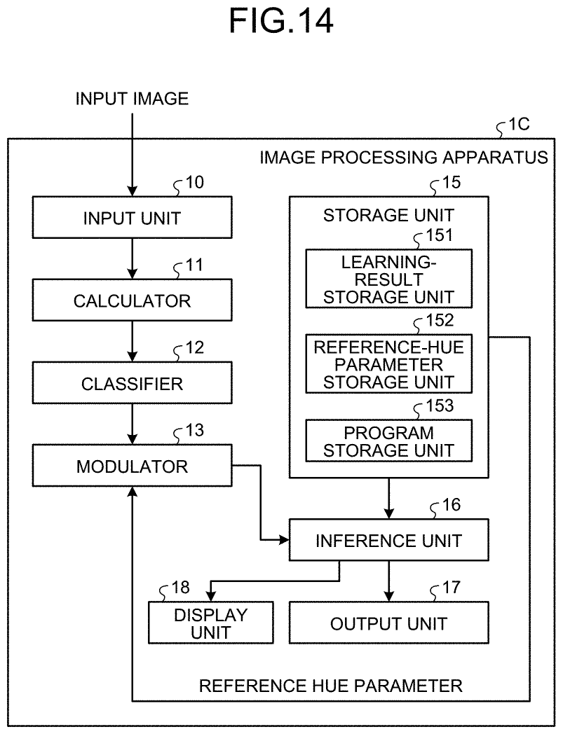

FIG. 14 is a block diagram illustrating a functional configuration of an image processing apparatus according to a fourth embodiment. An image processing apparatus 1C illustrated in FIG. 14 further includes a display unit 18 in addition to the configuration of the image processing apparatus 1A according to the second embodiment described above.

The display unit 18 displays the information and the image corresponding to various types of data output from the inference unit 16. The display unit 18 is configured by using a liquid crystal, an organic EL, or the like.

Process of Image Processing Apparatus

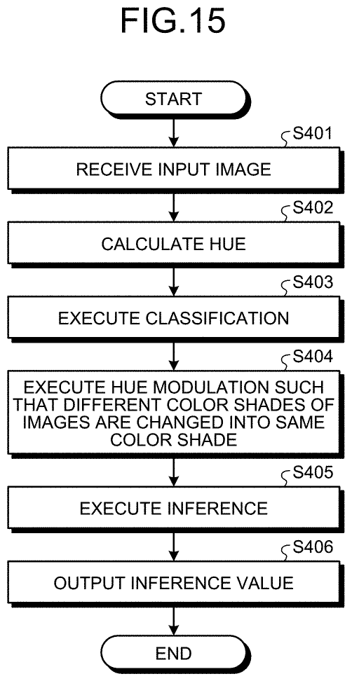

FIG. 15 is a flowchart illustrating the overview of a process performed by the image processing apparatus 1C according to the fourth embodiment. Steps S401 to S403, S405, and S406 correspond to Steps S201 to S203, S205, and S206, respectively, in FIG. 8 described above, and only Step S404 is different. Only Step S404 is described below.

At Step S404, the modulator 13 executes hue modulation on input images such that the different color shades of the images are changed into the same color shade. After Step S404, the image processing apparatus 1C proceeds to Step S405.

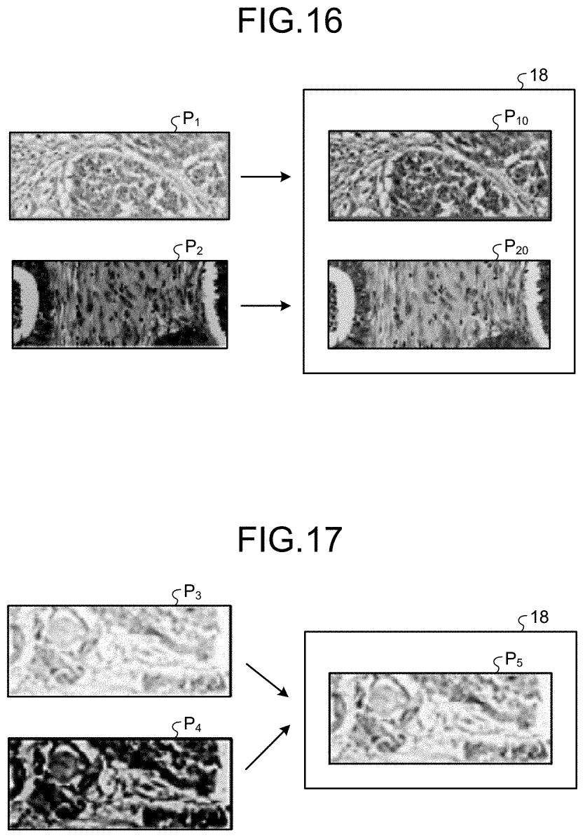

FIG. 16 is a diagram schematically illustrating an example of the image displayed by the display unit. FIG. 17 is a diagram schematically illustrating an example of another image displayed by the display unit.

As illustrated in FIG. 16, on a specimen image P₁ and a specimen image P₂ having different color shades due to the degree of staining, or the like, the modulator 13 executes hue modulation with the fixed value for the hue to generate an image P₁₀ and an image P₂₀ having a predetermined color shade and outputs the image P₁₀ and the image P₂₀ to the display unit 18. The display unit 18 displays the image Pic and the image P₂₀ side by side. Thus, the user may always observe an image having the same color shade so as to observe a structure, a state, and the like, in a stable manner.

As illustrated in FIG. 17, with regard to a specimen image P₄ having a different color shade from that of a specimen image P₃ due to the degree of staining, or the like, the modulator 13 sets the fixed value for the hue, generates an image P₅ having the same color shade as that of the specimen image P₃, and outputs the image P₅ to the display unit 18. The display unit 18 displays the specimen image P₃ and the image P₅ side by side. When the specimen images having different color shades are compared with each other, it may be difficult for the user to properly evaluate the specimen images; however, as the display unit 18 displays the specimen images, which have different color shades, in the same color shade, it is possible to simply observe and compare only the differences in the state of a cell and a tissue.

According to the fourth embodiment described above, as the display unit 18 displays the specimen images, which have different color shades, in the same color shade, it is possible to simply observe and compare only the differences in the state of a cell and a tissue.

### Fifth Embodiment

Next, a fifth embodiment of the present disclosure is described. An image processing apparatus according to the fifth embodiment is different from the image processing apparatus according to the second embodiment described above in the configuration and the process performed. Specifically, according to the fifth embodiment, the standard hue is calculated. After a configuration of the image processing apparatus according to the fifth embodiment is described, the process performed by the image processing apparatus according to the fifth embodiment is described below.

Configuration of Image Processing Apparatus

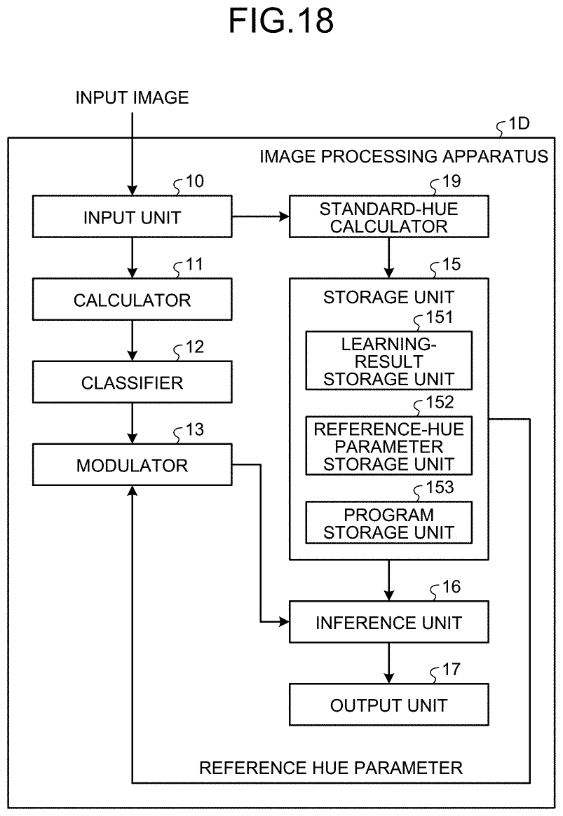

FIG. 18 is a block diagram illustrating a functional configuration of the image processing apparatus according to the fifth embodiment. An image processing apparatus 1D illustrated in FIG. 18 further includes a standard-hue calculator 19 in addition to the configuration of the image processing apparatus 1A according to the second embodiment described above.

The standard-hue calculator 19 calculates the color distribution of the prepared images for calculating a standard value to calculate a standard distribution.

Process of Image Processing Apparatus

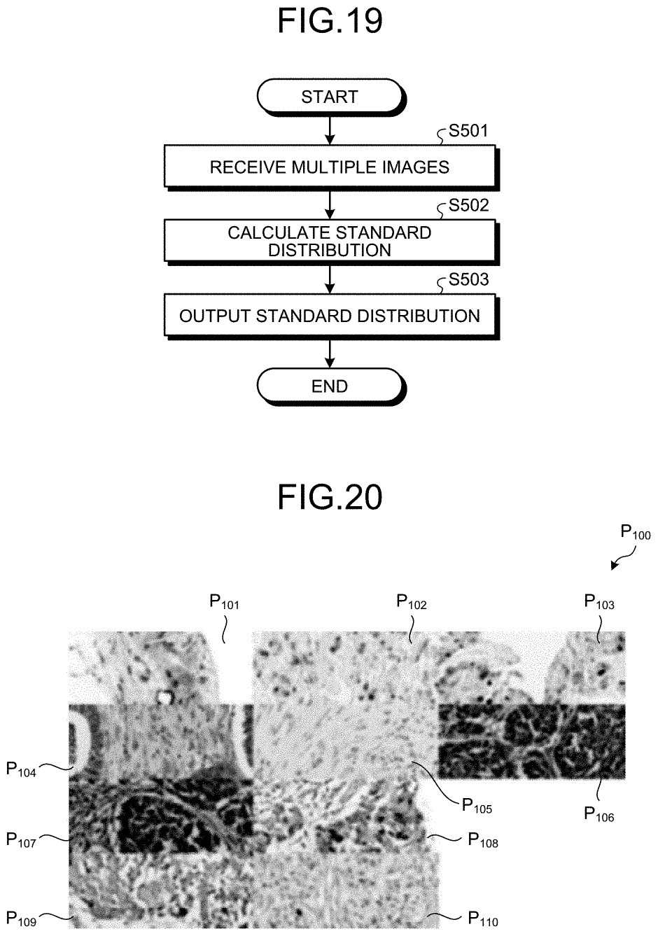

Next, a process performed by the image processing apparatus 1D is described. FIG. 19 is a flowchart illustrating the overview of the process performed by the image processing apparatus 1D.

As illustrated in FIG. 19, the input unit 10 first receives multiple images from outside (Step S501). FIG. 20 is a diagram schematically illustrating an example of the images input to the input unit 10. As illustrated in FIG. 20, the input unit 10 receives multiple images P101 to P110 from outside.

Then, the standard-hue calculator 19 calculates the color distributions of the images input from the input unit 10 to calculate the standard distribution (Step S502) and outputs the calculated standard distribution to the reference-hue parameter storage unit 152 of the storage unit 15 (Step 3503). After Step S503, the image processing apparatus 1D ends this process.

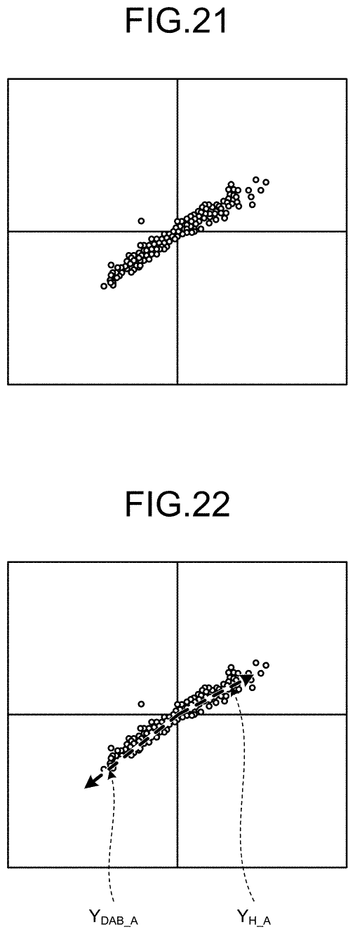

Here, the method for calculating the standard distribution by the standard-hue calculator 19 is described. FIG. 21 is a diagram schematically illustrating an example of the standard distribution by the standard-hue calculator 19. FIG. 22 is a diagram schematically illustrating an average hue axis. In FIG. 22, an arrow Y_{DAB_A} represents an average hue axis of the hue in the distribution regarded as DAB, and an arrow Y_{H_A} represents an average hue axis of the hue in the distribution regarded as H.

As illustrated in FIGS. 21 to 22, the standard-hue calculator 19 first combines all of the prepared images for calculating the standard value to calculate the color distribution of each pixel of a combined image P100 and sets the calculation result as the standard distribution. As illustrated in FIG. 22, the standard-hue calculator 19 sets, in the standard distribution, the average of the hue in the distribution regarded as DAB as the DAB average hue and the average of the hue in the distribution regarded as H as the H average hue. In FIG. 22, as indicated by the arrow Y_{DAB_A} and the arrow Y_{H_A}, the DAB average hue and the H average hue are the DAB average hue axis and the H average hue axis, respectively. The standard-hue calculator 19 sets the range of hue in the distribution range of DAB and H and generates a value within the range as the standard distribution. The standard distribution is used for the rotation and the fixed value for the hue described in the first embodiment to the fourth embodiment described above.

According to the fifth embodiment described above, the standard-hue calculator 19 sets, in the standard distribution, the average of the hue in the distribution regarded as DAB as the DAB average hue and the average of the hue in the distribution regarded as H as the H average hue so as to calculate the standard hue (the standard hue parameter).

### Sixth Embodiment

Next, a sixth embodiment of the present disclosure is described. An image processing apparatus according to the sixth embodiment is the same as that in the fifth embodiment described above in the configuration and is different in the process performed by the image processing apparatus. Specifically, according to the sixth embodiment, the color is modulated as appropriate for the trained learning unit. A learning method implemented by the learning unit included in the image processing apparatus according to the sixth embodiment is described below. The same components as those in the fifth embodiment described above are denoted by the same reference numeral, and the detailed description is omitted.

Learning Process by Learning Unit

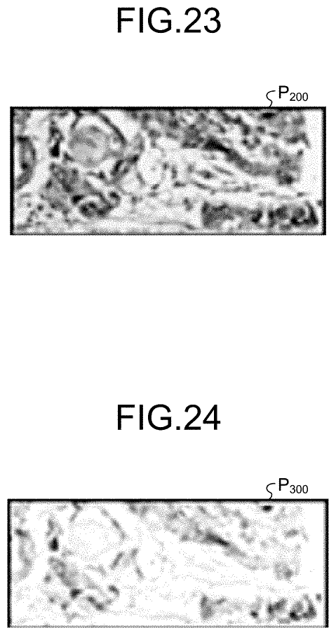

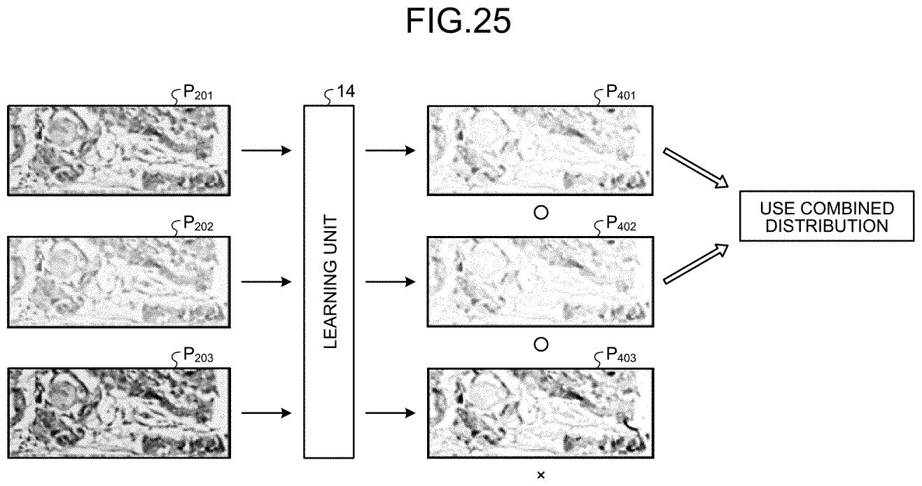

FIG. 23 is a diagram schematically illustrating an example of the input image to be learned by the learning unit 14. FIG. 24 is a diagram schematically illustrating an example of the correct image to be learned by the learning unit 14. FIG. 25 is a diagram schematically illustrating a learning process by the learning unit 14.

As the image used for learning is unknown, the learning unit 14 calculates the parameter for setting the appropriate color shade as described below. As illustrated in FIGS. 23 and 25, the standard-hue calculator 19 first generates multiple images P₂₁₀ to P₂₀₃ that are obtained by rotating the hue of an input image P₂₀₀ at different angles. As illustrated in FIG. 25, the standard-hue calculator 19 inputs the images P₂₀₁ to P₂₀₃ to the learning unit 14. Subsequently, the learning unit 14 outputs multiple output images P₄₀₁ to P₄₀₃ based on the images P₂₀₁ to P₂₀₃ and the learning result input from the standard-hue calculator 19. As illustrated in FIGS. 24 and 25, the user compares the output images P₄₀₁ to P₄₀₃ with the correct image P₃₀₀ and operates an operating unit (not illustrated) to select an output image whose error falls within an allowable range. Then, the standard-hue calculator 19 combines the color distributions of the input images of the output image P₄₀₁ and the output image P₄₀₂ selected by the user and calculates the average hue by using the same method as that described above in the fifth embodiment.

According to the sixth embodiment described above, it is possible to also input the image having the appropriately modulated color shade to the existing learning unit (learning device).

### Other Embodiments

The components described in the first embodiment to the sixth embodiment described above may be combined as appropriate to form various embodiments. For example, some components may be deleted from all the components described in the first embodiment to the sixth embodiment described above. Furthermore, the components described in the first embodiment to the fifth embodiment described above may be combined as appropriate.

In the first embodiment to the sixth embodiment, the “unit” described above may be replaced with a “means”, a “circuitry”, or the like. For example, the input unit may be replaced with an input means or an input circuitry.

A program to be executed by the image processing apparatuses according to the first embodiment to the sixth embodiment is provided by being recorded in a computer-readable recording medium such as a CD-ROM, a flexible disk (FD), a CD-R, a digital versatile disk (DVD), a USB medium, or a flash memory, in the form of file data installable or executable.

A configuration may be such that a program to be executed by the image processing apparatuses according to the first embodiment to the sixth embodiment is provided by being stored on a computer connected via a network, such as the Internet, and downloaded via the network. A program to be executed by the image processing apparatuses according to the first embodiment to the sixth embodiment may be provided or distributed via a network such as the Internet.

Although an input image is received from various devices via, for example, a transmission cable according to the first embodiment to the sixth embodiment, it does not need to be for example wired, and it may be wireless. In this case, a signal may be transmitted from each device in accordance with a predetermined wireless communication standard (e.g., Wi-Fi (registered trademark) or Bluetooth (registered trademark)). It is obvious that wireless communication may be executed in accordance with a different wireless communication standard.

In the flowcharts described in this description, the expressions such as “first”, “then”, and “subsequently” are used to indicate the order of processes at steps; however, the order of processes necessary to implement the present disclosure is not uniquely defined by the expressions. That is, the order of processes in the flowcharts in this description may be changed as long as there is no contradiction.

According to the present disclosure, there is an advantage such that it is possible to acquire different learning images in a simple process.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising a processor comprising hardware, the processor being configured to:
calculate a hue of each pixel of a stained image that is input from outside;
execute classification on each pixel of the stained image based on the hue;
modulate a color tone of the pixel of the stained image in each class having undergone the classification;
combine a plurality of input images to generate a combined image;
calculate a color distribution of each pixel of the combined image;
execute classification on each pixel of the combined image by using the color distribution; and
calculate an average hue of each class having undergone the classification on each pixel of the combined image based on the color distribution and a classification result of classification of the combined image so as to calculate a standard hue.

2. The image processing apparatus according to claim 1, wherein the processor is further configured to modulate an average value of a hue of each class having undergone the classification on each pixel of the stained image so as to be a hue that matches the calculated standard hue.

3. The image processing apparatus according to claim 1, wherein the processor is further configured to modulate hues of all pixels belonging to each class having undergone the classification on each pixel of the stained image so as to be a hue that matches the calculated standard hue.

4. The image processing apparatus according to claim 1, wherein the processor is further configured to store, in a storage, a learning result of learning based on the stained image having undergone hue modulation and on a correct value associated with the stained image.

5. The image processing apparatus according to claim 1, wherein the processor is further configured to execute inference based on a learning result stored in a storage and on the stained image having undergone hue modulation.

6. The image processing apparatus according to claim 5, further comprising a display configured to display an inference result that is inferred by the processor.

7. An image processing apparatus comprising a processor comprising hardware, the processor being configured to:
calculate a hue of each pixel of a stained image that is input from outside;
execute classification on each pixel of the stained image based on the hue;
modulate a color tone of the pixel of the stained image in each class having undergone the classification;
rotate a hue of an input image associated with a correct value at different rotation angles to generate a plurality of images having different hues;
generate, based on the plurality of images and on a learning result stored in a storage, a plurality of output images;
combine hue ranges of images whose error between the plurality of output images and the correct value falls within an allowable range to calculate a color distribution; and
calculate an average hue of each class having undergone classification on each pixel of the input image by using the color distribution so as to calculate a standard hue.

8. The image processing apparatus according to claim 7, wherein the processor is further configured to modulate an average value of a hue of each class having undergone the classification on each pixel of the stained image so as to be a hue that matches the calculated standard hue.

9. The image processing apparatus according to claim 7, wherein the processor is further configured to modulate hues of all pixels belonging to each class having undergone the classification on each pixel of the stained image so as to be a hue that matches the calculated standard hue.

10. The image processing apparatus according to claim 7, wherein the processor is further configured to store, in a storage, a learning result of learning based on the stained image having undergone hue modulation and on a correct value associated with the stained image.

11. The image processing apparatus according to claim 7, wherein the processor is further configured to execute inference based on a learning result stored in a storage and on the stained image having undergone hue modulation.

12. The image processing apparatus according to claim 11, further comprising a display configured to display an inference result that is inferred by the processor.

13. An image processing method implemented by an image processing apparatus, the image processing method comprising:
calculating a hue of each pixel of a stained image that is input from outside;
executing classification on each pixel of the stained image based on the hue;
modulating a color tone of the pixel of the stained image in each class having undergone the classification;
combining a plurality of input images to generate a combined image;
calculating a color distribution of each pixel of the combined image;
executing classification on each pixel of the combined image by using the color distribution; and
calculating an average hue of each class having undergone the classification on each pixel of the combined image based on the color distribution and a classification result of classification of the combined image so as to calculate a standard hue.

14. A non-transitory computer-readable recording medium with an executable program stored thereon, the program causing an image processing apparatus to execute:
calculating a hue of each pixel of a stained image that is input from outside;
executing classification on each pixel of the stained image based on the hue;
modulating a color tone of the pixel of the stained image in each class having undergone the classification;
combining a plurality of input images to generate a combined image;
calculating a color distribution of each pixel of the combined image;
executing classification on each pixel of the combined image by using the color distribution; and
calculating an average hue of each class having undergone the classification on each pixel of the combined image based on the color distribution and a classification result of classification of the combined image so as to calculate a standard hue.

15. An image processing method implemented by an image processing apparatus, the image processing method comprising:
calculating a hue of each pixel of a stained image that is input from outside;
executing classification on each pixel of the stained image based on the hue;
modulating a color tone of the pixel of the stained image in each class having undergone the classification;
rotating a hue of an input image associated with a correct value at different rotation angles to generate a plurality of images having different hues;
generating, based on the plurality of images and on a learning result stored in a storage, a plurality of output images;
combining hue ranges of images whose error between the plurality of output images and the correct value falls within an allowable range to calculate a color distribution; and
calculating an average hue of each class having undergone classification on each pixel of the input image by using the color distribution so as to calculate a standard hue.

16. A non-transitory computer-readable recording medium with an executable program stored thereon, the program causing an image processing apparatus to execute:
calculating a hue of each pixel of a stained image that is input from outside;
executing classification on each pixel of the stained image based on the hue;
modulating a color tone of the pixel of the stained image in each class having undergone the classification;
rotating a hue of an input image associated with a correct value at different rotation angles to generate a plurality of images having different hues;
generating, based on the plurality of images and on a learning result stored in a storage, a plurality of output images;
combining hue ranges of images whose error between the plurality of output images and the correct value falls within an allowable range to calculate a color distribution; and
calculating an average hue of each class having undergone classification on each pixel of the input image by using the color distribution so as to calculate a standard hue.
